(19) United States
(12) Patent Application Publication
(10) Pub. No.: US 2010/0305000 A1
(43) Pub. Date: Dec. 2, 2010

(54) METHOD AND USE OF MICROARRAY TECHNOLOGY AND PROTEOGENOMIC ANALYSIS TO PREDICT EFFICACY OF HUMAN AND XENOGRAPHIC CELL, TISSUE AND ORGAN TRANSPLANT

(75) Inventors: Aby J. Mathew, Vestal, NY (US); Robert Van Buskirk, Apalachin, NY (US); John G. Baust, Candor, NY (US); John M. Baust, Owego, NY (US); Dominic M. Clarke, Bothell, WA (US); Ian B. Nicoud, Seattle, WA (US)

Correspondence Address: BROWN & MICHAELS, PC;400 M & T BANK BUILDING, 118 NORTH TIOGA ST, ITHACA, NY, 14850, US

(73) Assignee: Biolife Solutions Inc., Bothell, WA (US)

(21) Appl. No.: 12/855,805

(22) Filed: Aug. 13, 2010

Related U.S. Application Data
(62) Division of application No. 12/147,884, filed on Jun. 27, 2008.
Application No. 12/147,884 is a continuation-in-part of application No. 10/372,579, filed on Feb. 21, 2003, now abandoned.
(60) Provisional application No. 60/358,386, filed on Feb. 22, 2002.

(51) Int. Cl. C40B 30/04; C12Q 1/02; C12Q 1/68

(52) U.S. Cl. 506/9; 435/29; 435/6

(57) ABSTRACT

The present invention is directed to systems and proteogenomic methods for predicting the success of the transplant of a cell, tissue, or organ by providing a means to determine the quality of the cell, tissue, or organ to be transplanted. In one embodiment, the present invention uses samples from the preservation solution to obtain phenomic fingerprints correlated with transplant pre-operative and post-operative data as a pre-operative tissue diagnostic and procedural success predictive indicator.

### REFERENCE TO RELATED APPLICATIONS

This is a continuation-in-part patent application of copending application Ser. No. 10/372,579, filed Feb. 21, 2003, entitled “Method and use of protein microarray technology and proteomic analysis to determine efficacy of human and xenographic cell, tissue and organ transplant”, which claims one or more inventions which were disclosed in Provisional Application No. 60/358,386, filed Feb. 22, 2002, entitled “Method and use of protein microarray technology and proteomic analysis to determine efficacy of human and xenographic cell, tissue and organ transplant”. The benefit under 35 USC §119(e) of the United States provisional application is hereby claimed, and the aforementioned applications are hereby incorporated herein by reference.

### BACKGROUND OF THE INVENTION

1. Field of the Invention

The present invention relates to tools and methods to improve success of a cell, tissue, or organ transplant.

2. Description of Related Art

There are many types of evaluations and tests used in the cell, tissue, and organ transplantation process. Pre-operative tests focus on the overall health of the transplant recipient, and may include electrocardiograms and echocardiograms to evaluate cardiac status, blood tests for tissue typing and to determine that the patient is free of infection or other conditions (e.g., cancer) that would contraindicate transplantation, and tests to evaluate the patient's immune status. Ultrasound images may also be taken to check for overall health, or for the condition of areas of the body relating to the transplant site. For example, a kidney transplant recipient may undergo abdominal and renal ultrasounds to check the abdominal area, the gall bladder, and the kidneys.

Post-operative testing focuses on identifying rejection of the cells, tissues, or organs that were transplanted. Blood tests and biopsies assist in evaluating the health and function of the new cells, tissues, or organs as well as the health of the transplant recipient. If the patient exhibits signs of a rejection episode, changes to the immunosuppressive regimen must be made, and in some cases the patient may require removal of the transplant and re-transplantation with new donor material.

Microarray technology is being used in a number of ways to study DNA, RNA, and proteins, including protein-protein interactions, protein reactions with drugs, and the quantity of various proteins in a sample.

DNA microarrays are used for gene expression profiling, determining DNA-protein binding domains, and have been applied to determine predisposition to disease and to identify drug candidates. Protein microarrays are similar in their use but identify changes at the protein level, including protein-protein interactions, protein reactions with drugs, and the quantity of various proteins in a sample.

While DNA sequences have relatively ubiquitous expression, the expression of proteins can differ from cell to cell and over time due to environmental changes and interactions with other cells. Determining the quantity of proteins in a sample is achieved through the use of arrays coated with capture agents that bind with the proteins in the sample. Analysis of the amount and location of the bound proteins on the array can be used in a variety of proteomic research approaches.

Von Eggeling, et al. (2000, BioTechniques 29: 1066-1070) reported the utilization of ProteinChip® (Ciphergen, Fremont, Calif.) microarray technology for the analysis of cancerous tissue protein profiles. That study described the use of protein microarray analysis for distinguishing between cancerous and normal tissue. The ProteinChip® technology has also been used as a diagnostic tool to screen urine in order to assess renal dysfunction following administration of radiocontrast medium for cardiac function imaging (Hampel, et al. (2001, J. Am. Soc. Nephrol. 12: 1026-1035).

Other reports on the utilization of protein microarray technology for the identification of candidate genes involved in tissue repair/regeneration, disease diagnosis, as well as cancer biomarker identification further support the role of high-through put protein analysis in research and clinical settings (Li e al., 2000, Biochim. Biophys. Acta 1524: 102-109; Tonge et al., 2001, Proteomics 1: 377-396; Vlahou et al., 2001, Am. J. Pathol. 158: 1491-1502).

In 2005, Expression Diagnostics (XDx, Brisbane, Calif.) introduced the AlloMap® gene chip to identify patients at risk or not at risk for cardiac allograft rejection, by identifying genomic changes in a distinct population of DNA targets. Using 9 genes for reproducibility and standardization and 11 identifying genes in 7 diverse molecular pathways of the immune system, AlloMap® testing yields a test score ranging from 0 (very low risk of rejection) to 40 (higher risk for rejection). These targets include identification of macrophage activation, platelet activation, hematopoiesis, T-cell activation and regulation, and steroid responsiveness.

U.S. Patent Publication No. 2007/0082356, METHODS OF EVALUATING TRANSPLANT REJECTION, by Strom et al., herein incorporated by reference, provides methods for the post-operative detection of transplant rejection by monitoring the upregulation in gene expression of two or more selected genes. These genes may include immune activation genes such as, perforin, granzyme B, FAS ligand, or cytoprotective genes such as heme oxygenase-1 and A20.

A DNA microarray is a high-throughput technology, which includes an arrayed series of thousands of microscopic spots of DNA oligonucleotides. Each spot (also known as a feature) contains a specific DNA sequence, which may be a short section of a gene or other DNA element that is used as a probe to hybridize a cDNA or cRNA sample under high-stringency conditions. The sample is called a target. One way to detect and quantify probe-target hybridization is by fluorescence-based detection of fluorophore-labeled targets to determine relative abundance of nucleic acid sequences in the target.

In Southern blotting, a mix of DNA fragments are attached to a substrate and then probed with a tagged gene or fragment of known origin. The tags include, but are not limited to, a radioactive tag, a chemiluminescent, or a fluorophor tag. DNA microarray technology, including the use of miniaturized microarrays for gene expression profiling, evolved from Southern blotting. Arrays of DNA can be spatially arranged, for example in the well known “gene chip”. Alternatively, the arrays may be specific DNA sequences labeled so that they may be independently identified in solution. A traditional solid-phase array includes a solid surface onto which a collection of microscopic DNA spots are attached. Some examples of the solid surface include, but are not limited to, glass, plastic or silicon biochips. Thousands of the affixed DNA segments, known as probes, may be placed in known locations on a single DNA microarray.

### SUMMARY OF THE INVENTION

The present invention is directed to systems and proteogenomic methods for predicting the success of the transplant of a cell, tissue, or organ by providing a means to determine the quality of the cell, tissue, or organ to be transplanted.

In one embodiment, the present invention uses samples from the preservation solution to obtain phenomic fingerprints correlated with transplant pre-operative and post-operative data as a pre-operative tissue diagnostic and procedural success predictive indicator.

### BRIEF DESCRIPTION OF THE DRAWINGS

FIG. 1 shows a schematic diagram of an apparatus for assessing the status of a cell, tissue or organ before transplant.

FIG. 2 shows a schematic diagram of one example of a method of generating a protein difference map.

FIG. 3 shows protein spectra of purified Insulin and Glucagon protein standards analyzed on Normal Phase 1 (NP1) protein chip arrays. Standard analysis was performed as a means of assessing the accuracy of the ProteinChip® system in comparison with reported molecular weight values. In addition, Insulin standards (20 fmol) were analyzed to determine detection variation within and between array spots on the NP1 chips. Glucagon standards were spotted in varying concentrations (6 and 20 fmol) to determine the sample detection sensitivity of the protein chips.

FIG. 4 shows protein spectra obtained from analysis of preservation medium at various time points during preservation. Analysis of fresh and transport preservation medium (Spectra A and B, respectively) revealed a relative flat line spectra pattern indicating minimal protein presence. Analysis of preservation medium flushed from kidneys revealed the presence of a substantial amount of protein present in the solution, which continued to increase, as well as the development of new protein peaks as the preservation interval extended.

FIG. 5 shows protein spectra of urinary cellular lysate samples obtained from renal transplant donor and recipient patients prior to (donor) and following (recipient) successful transplantation. Donor analysis yielded a base line profile for comparative purposes. Analysis of recipient patient samples revealed an increase in the profile intensity correlating to an increase in protein expression 24 hours and the appearance of unique proteins 48 hours after transplantation. Continued analysis at 72 hours revealed a marked decrease in protein levels that represented a return to levels similar to that of the initial donor profile.

FIG. 6 shows a schematic diagram of a process performed by a computerized system for identifying the condition of a cell, tissue or organ that is being considered for transplant. A set of stored biomarker data for the cell, tissue or organ to be assessed, or a specific subset of stored biomarker data is chosen.

FIG. 7 shows a schematic of a computer display screen shot including a graphic representation of buttons to specify biomarker(s) to be assessed, start assessment and set comparison process options.

FIG. 8 shows a schematic of a computer display screen shot displaying comparison process options.

### DETAILED DESCRIPTION OF THE INVENTION

The existing mechanism for determining the suitability of a tissue-matched organ for transplant relies to a great extent on imprecise analyses of the general “look and feel” of the organ, criterion highly dependent on the experience of the individuals performing the analysis. That is, in many cases the diagnostic tools utilized to assess organ quality prior to transplantation rely on a physical assessment of the tissue by the physician prior to implantation (Brasile et al., 2001, Clin. Transplant. 15: 369-374). This physical assessment typically includes evaluating organ color, rigidity, temperature, clarity of preservation solution, etc., and often results in underutilization based on nonfunctional conclusions (Pokorny et al., 1999, Transplant. Proc. 31: 2074-2076). This assessment regime serves as an unofficial standard due to limitations in availability of more quantitative diagnostic technologies. The methods and apparatus disclosed herein permit a rapid, real-time analysis of transplant status both before and after transplantation, thereby providing guidance on pre- and post-transplant decision making.

The present invention provides methods for evaluating the medical condition of a cell, tissue or organ before or after it is transplanted. A plurality of biomarkers for the status of the cell, tissue or organ are detected using an array, preferably a microarray, and the presence, absence or relative amounts of those biomarkers are compared with a reference value or a biomarker difference map. The reference represents biomarkers for that cell, tissue or organ from pre- and/or post transplant cells, tissues or organs for which clinical outcomes, positive or negative, are known. The comparison of the markers or their pattern guides clinical decision-making in the transplant process.

The present invention also provides an apparatus for predicting the success of the transplant of a cell, tissue or organ. The apparatus preferably includes a platform or holder to hold surface chemistry or a capture agent necessary to detect a plurality of different biomarkers in a sample, a detection mechanism to determine the quantity and/or type of biomarkers bound to the platform, a processor including a comparison mechanism for comparing biomarker detection data from the sample with a reference and a mechanism for determining the condition of the cell, tissue, or organ to be transplanted based on the comparison of biomarker detection data from the sample with the reference.

In prior art transplantation procedures, the preservation/perfusion solution in which a cell, tissue, or organ is stored was not analyzed for biomarkers or used as a predictor of transplant success. In contrast, the present invention uses samples from the preservation solution to obtain phenomic fingerprints correlated with transplant pre-operative and post-operative data as a pre-operative tissue diagnostic and predictive indicator of procedural success.

“Proteogenomics”, as defined herein, is the study of both proteomics (proteins) and genomics (genes).

A “peptide”, as defined herein, is any of various natural or synthetic compounds containing two or more amino acids linked by the carboxyl group of one amino acid and the amino group of another, and includes both polypeptides and proteins.

As used herein, the “medical condition” of a cell, tissue, or organ to be used as a transplant for a recipient in need of transplantation therapy is defined as the quality of the cell, tissue, or organ, and whether or not it is suitable for transplant in the recipient.

As used herein, a sample is “from a cell, tissue, or organ” if it is taken directly from the cell, tissue or organ, if it is obtained from a body fluid (e.g., serum or urine) of an individual including that cell, tissue or organ, or if it is taken from fluid in which the cell, tissue or organ was or is stored prior to transplant.

As used herein, a “reference pattern” is a pattern of a plurality of biomarkers that is created using samples from pre or post transplant cells, tissues or organs associated with transplant recipients for which a clinical outcome is known. The reference pattern preferably includes a correlation between a positive outcome or a negative outcome and the absence, presence or amount of a plurality of biomarkers collected during the course of the transplant procedure from donor harvest to recipient transplantation.

As used herein, “a difference between the pattern observed for a transplant and a reference pattern” encompasses both similarities and differences between biomarker patterns. Thus, when there is no difference or very little difference between a reference pattern and a test sample pattern, the “difference” is indicative that the transplant outcome for the test sample will be similar to the outcome for the reference sample(s). Alternatively, where there is a wide “difference” (e.g., 50% or more higher or lower than the reference), the outcome of the test sample transplant will likely differ from the outcome of the reference pattern sample(s).

As used herein, the term “clinical outcome” is the eventual medical success or failure of transplant therapy in the context of a given patient. Clinical outcome represents an extrinsic piece of medical data associated with the success or failure of a particular patient's medical treatment. Some indicators of clinical outcome include, but are not limited to, warm-ischemic time of the graft, cold-ischemic time of the graft, intra-operative complications, length of surgical procedure, patient survival, number of days in the hospital prior to release, post-operative complications, rejection of the transplant, immunosuppressive regimen (drug type and dose), quality of life (QOL), and level of monitoring required after the transplant. A positive clinical outcome is post-transplant success including, but not limited to, a non-rejected transplant, healthy post-transplant function of the graft, successful immunosuppressive regimen, and improved QOL. Negative clinical outcomes include, but are not limited to, graft rejection (hyper-acute, acute, chronic), organ failure, re-transplantation, decreased QOL, and death.

The present invention uses microarray technology to obtain a biomarker pattern for the cell, tissue, or organ that is to be used in the transplant. The biomarkers include, but are not limited to, DNA, RNA, peptides, and biomarkers associated with apoptosis and necrosis. A sample is placed in a system including a holder. In one embodiment, the holder is a microarray chip coated with capture agents, which are preferably surface-enhanced. The biomarkers in the sample bind to certain capture agents on the platform. Combined with a detection mechanism, the amount of each of the relevant biomarkers in the sample can be quantified to generate a biomarker pattern. A reference pattern or biomarker difference map may be created using the same technique, comparing the biomarker patterns of a healthy transplant to a rejected transplant. The comparison includes a measurement of the presence, absence, or amount of the plurality of biomarkers in the two samples.

In one embodiment, the sample includes a sample of the solution used to store and/or transport the cell, tissue, or organ to be transplanted. In another embodiment, the sample includes a fluid sample from the patient who has received the cell, tissue, or organ.

In one embodiment, the holder may be any holder including, but not limited to, a planar surface, a bead, a cylinder or a microarray. In another embodiment, the measurement is performed using a nucleic acid array. The nucleic acid array is preferably a gene chip or a microarray. In another embodiment, proeogenomic analysis is used for the measurement.

In another embodiment, the surface chemistry or capture agent includes an antibody. In yet another embodiment, the surface chemistry includes an ion exchange or reversed-phase affinity agent.

In one embodiment, the detection mechanism includes Surface-Enhanced Laser Desorption/Ionization Time-Of-Flight (SELDI TOF). In another embodiment, the detection mechanism includes a labeled antibody. In yet another embodiment, the detection mechanism includes surface plasmon resonance.

The present invention also provides an apparatus and method for determining the quality of a donor cell, tissue, or organ. The method includes collection of a sample of the perfusion/preservation solution contained within or around the donor cell, tissue, or organ, and detecting a plurality of different biomarkers within the sample. The biomarkers include, but are not limited to, DNA, RNA, peptides, and biomarkers associated with apoptosis and necrosis. A holder preferably holds at least one of a surface chemistry and/or a capture agent necessary to detect a plurality of different biomarkers within the sample. A detection mechanism determines biomarker detection data including at least one of quantity and type of biomarkers bound to the holder.

In one embodiment, the sample includes a sample of the solution used to stored and/or transport the cell, tissue, or organ to be transplanted. In another embodiment, the sample includes a fluid sample from the patient who has received the cell, tissue, or organ.

In one embodiment, the holder may be any holder including, but not limited to, a planar surface, a bead, a cylinder or a microarray. In another embodiment, the measurement is performed using a nucleic acid array. The nucleic acid array is preferably a gene chip or a microarray. In another embodiment, proteogenomic analysis is used for the measurement.

In another embodiment, the surface chemistry or capture agent includes an antibody. In yet another embodiment, the surface chemistry includes an ion exchange or reversed-phase affinity agent.

In another embodiment, the detection mechanism includes Surface-Enhanced Laser Desorption/Ionization Time-Of-Flight (SELDI TOF). In another embodiment, the detection mechanism includes a labeled antibody. In yet another embodiment, the detection mechanism includes surface plasmon resonance.

Another apparatus and method of the present invention predict success of a transplanted cell, tissue, or organ. The method includes collecting a sample of the perfusion/preservation solution within or surrounding the cell, tissue, or organ to be transplanted. The apparatus includes a holder to hold at least one of a surface chemistry and a capture agent necessary to detect a plurality of different biomarkers of a sample and a detection mechanism to determine biomarkers detection data including at least one of quantity and type of biomarkers bound to the holder. The apparatus also includes a processor with a comparison mechanism to compare the biomarker detection data from the sample with a reference pattern or a biomarker difference map; and a protocol for making treatment decisions based on the quality of the cell, tissue, or organ to be transplanted. The biomarkers include, but are not limited to, DNA, RNA, peptides, and biomarkers associated with apoptosis and necrosis.

In one embodiment, the sample includes a sample of the solution used to store and/or transport the cell, tissue, or organ to be transplanted. In another embodiment, the sample includes a fluid sample from the patient who has received the cell, tissue, or organ.

In one embodiment, the holder may be any holder including, but not limited to, a planar surface, a bead, a cylinder or a microarray. In another embodiment, the measurement is performed using a nucleic acid array. The nucleic acid array is preferably a gene chip or a microarray. In another embodiment, proteogenomic analysis is used for the measurement.

Another method of the present invention evaluates the medical condition of a cell, tissue, or organ to be used as a transplant. The method provides a cell, tissue or organ to be transplanted and uses a biomarker array to measure the amount of a plurality of biomarkers in a sample from the cell, tissue or organ, thereby determining a pattern. The pattern of the plurality of biomarkers from the cell, tissue, or organ is compared to the values for a reference pattern of the plurality of biomarkers. A difference between the pattern observed for the transplant and the reference pattern is indicative of the medical condition of the transplant. In a preferred embodiment, a polypeptide array is used to determine a pattern of a plurality of polypeptides. In other embodiments, nucleic acid arrays may be used to determine a pattern of a plurality of DNA or RNA. The nucleic acid array is preferably a gene chip or a microarray. In another embodiment, proteogenomic analysis is used for the measurement. The cell, tissue or organ may be tissue-matched, or the method may alternatively include the additional step of performing matching to assess a transplant donor-to-recipient match. In one embodiment, the comparing step includes measurement of the presence, absence, or amount of the plurality of biomarkers. In another embodiment, there are at least four biomarkers. In another embodiment, the measurement is performed using a protein array. In a preferred embodiment, the protein array is a microarray including a plurality of antibodies, an ion exchange affinity agent or a reversed-phase affinity agent.

In one embodiment, the sample includes a sample of the solution used to store and/or transport the cell, tissue, or organ to be transplanted. In another embodiment, the sample includes a fluid sample from the patient who has received the cell, tissue, or organ.

Another method of the present invention generates a biomarker difference map. A large collection of data related to biomarker expression across transplant material at a variety of stages of transplantation (donor, transport, post-transplant) is preferably gathered and changes in biomarker expression in correlation with clinical outcomes for any given transplant is analyzed. Reviewing and comparing expression patterns at any given point in the transplant process to known clinical outcomes creates a continuous reference pattern. The reference pattern then is the basis from which one can compare a sample of interest in a known stage in the transplant process to in order to predict the outcome. This permits samples from either the same or different stages to be compared.

A first biomarker pattern is preferably identified from a first cell, tissue, or organ and a second biomarker pattern is identified from a second cell, tissue, or organ is identified. The first and second biomarker patterns are compared, thereby generating a biomarker difference map. In one embodiment, the steps of identifying a biomarker pattern for each of the first cell, tissue or organ and the second cell, tissue, or organ include measuring the presence, absence, or amount of a plurality of biomarkers in a sample. The biomarkers include, but are not limited to, DNA, RNA, peptides, and biomarkers associated with apoptosis and necrosis. In one embodiment, the biomarker difference map is a protein difference map. In another embodiment, the first and second biomarker patterns include information regarding at least four biomarkers. In another embodiment, the biomarker pattern is identified using a microarray. In another embodiment, the first cell, tissue, or organ is the same type of cell, tissue, or organ as the second cell, tissue or organ. In another embodiment, the first biomarker pattern is derived from a healthy transplant and the second biomarker pattern is derived from a rejected transplant.

Another method of the present invention predicts the suitability of a cell, tissue, or organ for transplant. The method includes measuring the presence, absence, or amount of a plurality of biomarkers in a cell, tissue, or organ being evaluated for transplant, to generate a biomarker pattern, and comparing the biomarker pattern to a biomarker difference map representing the differences in presence, absence, or amount of the plurality of biomarkers exhibited in healthy versus unhealthy cells, tissues, or organs of the same kind, where the comparing step predicts the suitability of the cell, tissue, or organ. The biomarkers include, but are not limited to, DNA, RNA, peptides, and biomarkers associated with apoptosis and necrosis. In a preferred embodiment, the biomarkers are polypeptide biomarkers. In another preferred embodiment, the measuring step is performed using a microarray. The microarray preferably includes a plurality of antibodies and there are preferably at least four biomarkers.

In another embodiment, a biomarker difference map for evaluating materials for transplant, made as disclosed herein, is provided. In a preferred embodiment, the biomarker difference map is a protein difference map.

The biomarkers in the present invention include, but are not limited to, DNA, RNA, peptides, and biomarkers associated with apoptosis and necrosis. In one embodiment, the plurality of biomarkers associated with apoptosis and necrosis, include, but are not limited to, DNA, RNA, or peptides of caspases, cytochrome c, calpains, or the BAX family of proteins, for any cell, tissue, or organ.

In one embodiment, the cell, tissue, or organ is a kidney, and the biomarker pattern includes DNA, RNA, or peptide information regarding one or more of albumin, IgA, IgGm urokinase, thyroxine binding globulin, transferrin, anti-thrombin 3, protein S, protein C, amylase, chlecalcitol, Bence Jones protein, ribonuclease, and hemoglobin.

In another embodiment, the cell, tissue, or organ is a liver, and the biomarker pattern includes DNA, RNA, or peptide information regarding one or more of aspartate aminotransferase, alanine aminotransferase, bilirubin, glutamate dehydrogenase, malate dehydrogenase, ketose-1-phosphate aldolase, and lactate dehydrogenase.

In another embodiment, the cell, tissue, or organ is a heart, and the biomarker pattern includes DNA, RNA, or peptide information regarding one or more of creatine kinase, aspartate amino transferase, lactic acid dehydrogenase, and fructose aldolase.

In another embodiment, the cell, tissue, or organ is a pancreas or pancreatic islet cell, and the biomarker pattern includes DNA, RNA, or peptide information regarding one or more of amylase, lipase, aspartame aminotransferase, alanine aminotransferase, lactic acid dehydrogenase, alkaline phosphatase, leucine amidopeptidase, insulin, proinsulin, and glucose phosphate isomerase.

In another embodiment, the cell, tissue, or organ is a kidney, and the plurality of biomarkers includes DNA, RNA, or peptide information regarding one or more of albumin, IgA, IgGm urokinase, thyroxine binding globulin, transferrin, anti-thrombin 3, protein S, protein C, amylase, chlecalcitol, Bence Jones protein, ribonuclease, and hemoglobin.

In another embodiment, the cell, tissue, or organ is a liver, and the plurality of biomarkers includes DNA, RNA, or peptide information regarding one or more of aspartate aminotransferase, alanine aminotransferase, bilirubin, glutamate dehydrogenase, malate dehydrogenase, ketose-1-phosphate aldolase, and lactate dehydrogenase.

In another embodiment, the cell, tissue, or organ is a heart, and the plurality of biomarkers includes DNA, RNA, or peptide information regarding one or more of creatine kinase, aspartate amino transferase, lactic acid dehydrogenase, and fructose aldolase.

In another embodiment, the cell, tissue, or organ is a pancreas or pancreatic islet cell, and the plurality of biomarkers includes DNA, RNA, or peptide information regarding one or more of amylase, lipase, aspartame aminotransferase, alanine aminotransferase, lactic acid dehydrogenase, alkaline phosphatase, leucine amidopeptidase, insulin, proinsulin, and glucose phosphate isomerase.

In other embodiments, the cell, tissue or organ is lung, skin graft, neural tissue, limbs for reattachment, cornea, hair follicles, heart valves, cartilage or orthopedic tissues.

In one embodiment, the sample includes a sample of the storage/preservation solution used to store and/or transport the cell, tissue, or organ to be transplanted. In another embodiment, the sample includes a fluid sample from the patient who has received the cell, tissue, or organ.

In one embodiment, the source of the sample is urine, serum, plasma or saliva from a transplant donor or transplant recipient, or storage fluid for a cell, tissue or organ to be transplanted. In another embodiment, the source is one of a transplant recipient, a transplant donor, a cell to be transplanted, a tissue to be transplanted, an organ to be transplanted, a transplanted cell, a transplanted tissue, and a transplanted organ. In another embodiment, the indication of a likelihood of a successful transplant further provides a suggested transplant approach. Some suggested transplant approaches include, but are not limited to, a suggestion to proceed with the transplant with standard monitoring, a suggestion to proceed with the transplant with heightened monitoring, or a suggestion not to proceed with the transplant.

Biomarkers

An important aspect of transplant evaluation is the identification of biomarkers present in pre-transplant tissues or organs that correlate with post-transplant difficulties. Thus, the identification of biomarkers that predict later problems can aid the physician in determining whether or not to go forward with a transplant, or can guide their post-operative treatment by highlighting potential problems at an early stage.

Current technology for transplant monitoring relies on indicators of complications that are sometimes not apparent for days or weeks after the transplant. In contrast, the methods disclosed herein measure biomarkers before and immediately after transplantation, e.g., within minutes or hours (e.g., 1, 2, 4, 8, 12, 24, 36 or 48 hours) after transplantation. The identification of changes in one or more known or unknown biomarkers in this time frame provides a reference or biomarker difference map that is more reflective of the pre-transplant sample. Thus, rapid real-time monitoring of the quality of the donor material can provide the physician with accurate information with which to make decisions related to either the operation itself or through modification of post-operative therapeutic regimes, thereby reducing or eliminating the complications associated with many transplantation procedures.

The present invention evaluates post-transplant patient samples, body fluids or biopsies at a range of timepoints. These timepoints may be “early” timepoints, which occur within minutes or hours from the time of the transplant or “late” timepoints, which occur within days to weeks after the transplant. This evaluation identifies potential biomarkers, and correlates these biomarkers with clinical outcomes to identify target biomarkers for use in the predictive analysis. The post-transplant patient samples may also be used as part of the reference pattern for the particular biomarker or biomarkers being examined.

Methods disclosed herein identify and use biomarkers that indicate the status of a transplant. A “biomarker,” as the term is used herein, includes DNA, RNA, or a peptide that is an indicator for the status of a cell, tissue or organ transplant. The presence, absence or amount of the biomarker in the transplant or in a body fluid of a donor or recipient correlates with an aspect of the health or function of the transplant. Biomarkers include, but are not limited to, DNA, RNA, and peptides. In one embodiment, the plurality of biomarkers are associated with apoptosis and necrosis, and preferably include, but are not limited to, DNA, RNA, or peptides of caspases, cytochrome c, calpains, or the BAX family of proteins, for any cell, tissue, or organ.

In one embodiment, a biomarker is a known DNA, RNA, and/or peptide that indicates the status of a transplant. For example, the presence and amount of a known biomarker that becomes detectable in urine, serum or other fluid only when a transplant is under stress indicates that the cell, tissue or organ is stressed.

In some embodiments, patterns from more than one type of biomarker may be used to provide the most useful information for predicting the efficacy of human and xenographic cell, tissue and organ transplants. For example, one may choose to use both RNA and DNA biomarkers, both RNA and peptide biomarkers, both DNA and protein biomarkers, or RNA, DNA, and peptide biomarkers.

Examples of biomarkers that alone or together indicate the status of tissues or organs for transplant are described below. One or more of these biomarkers can be monitored relative to their presence, absence or amount in samples from healthy, non-transplanted individuals to evaluate the status of a given transplant before or after implantation. It is important to note that, as the technology described herein becomes adopted, biomarkers in addition to those discussed herein will be discovered. Use of these additional biomarkers is within the spirit of the present invention.

Some genes that correlate with the status of a transplant may include, but are not limited to, immune activation genes such as, perforin, granzyme B, FAS ligand, or cytoprotective genes such as heme oxygenase-1 and A20.

Because of its function, urine is a particularly appropriate fluid to measure the status of a transplant kidney. In healthy individuals, the protein content of urine is very low, so detection of increased proteinuria is itself indicative of stress to the organ. However, biomarkers that correlate with the status of the tissue include, for example, albumin, IgA, IgG, urokinase, thyroxine binding globulin, transferrin, anti-thrombin-3, protein S, protein C, amylase, chlecalcitol, Bence Jones protein, ribonuclease and hemoglobin.

The serum levels of the following polypeptides provide examples of biomarkers for the status of liver tissue before or after transplant: aspartate aminotransferase, alanine aminotransferase, bilirubin, glutamate dehydrogenase, malate dehydrogenase, ketose-1-phosphate aldolase and lactate dehydrogenase.

The serum levels of the following polypeptides provide examples of biomarkers for the status of cardiac tissue before or after transplant: creatine kinase, aspartate aminotransferase, lactic acid dehydrogenase and fructose aldolase.

The serum levels of the following polypeptides provide examples of biomarkers for the status of the pancreas, pancreatic islets or tissue before or after transplant: amylase, lipase, aspartame aminotransferase, alanine aminotransferase, lactic acid dehydrogenase, alkaline phosphatase, leucine aminopeptidase, insulin, proinsulin, and glucose phosphate isomerase.

The known biomarkers can be detected, for example, following their capture with specific antibodies immobilized on an array surface. Numerous antibodies are commercially available. Alternatively, one skilled in the art can generate a monoclonal or polyclonal antibody preparation suitable for capture of a known polypeptide. Alternatively, the molecular mass of the known biomarkers is known, permitting their detection in a sample by mass spectrometry. For DNA and RNA biomarkers, a nucleic acid array may be used. Some examples for the nucleic acid array include gene chips or microarrays.

Alternatively, the identity of the biomarker need not be known for it to be useful as a biomarker. The present invention includes specific arrays to identify target biomarkers in pre-transplant samples of preservation solution. In one embodiment, SELDI TOF arrays, which are further discussed below, are used. Potential biomarkers may alternatively be identified by evaluating the preservation solution from numerous transplants, correlating the samples to expression of biomarkers post-transplant, and correlating the overlapping biomarkers with clinical outcomes.

A sample from a transplant donor, recipient, or from the tissue itself (e.g., from hypothermic storage fluid) is evaluated for the presence and/or amount of an unknown biomarker that correlates with the status of the transplant.

To establish the ability to use unknown proteins as biomarkers, one can perform detection of proteins bound to a surface chemistry agent that binds a number of proteins, for example, an anion exchange agent. The bound proteins are then detected, for example by SELDI-TOF mass spectrometry, which generates a series of peaks corresponding to the molecular masses and amounts of the various proteins in the sample. The series of peaks provides a profile for that sample. The profiles of a number of samples from healthy donors and from transplant recipients in various stages of successful and unsuccessful transplant are then compared to identify peaks and patterns of peaks that correlate with the status of the transplant. Thus, the peaks and the proteins they represent, even though unknown, provide biomarkers for the status of the transplant. Of course, when an unknown biomarker is found to correlate closely with the status of a transplant, efforts can be focused on determining the identity of the biomarker protein, such that it can be further studied or even used as a known biomarker. Proteolytic peptide analysis and mass spectrometry can be used to identify the protein, as can microsequencing technology. For unknown DNA biomarkers, a DNA microarray could be used to identify an unknown DNA biomarker with the advancement of the human genome project.

For all aspects described herein, it is assumed that a donor cell, tissue or organ to be used as a transplant has been tissue matched with the recipient. This standard process of evaluating the immunological compatibility of the donor and recipient is very well known in the art.

Samples

Any biological fluid can be monitored for biomarkers, but as noted above, samples to monitor the status of a transplant will frequently be derived from urine or blood serum or plasma of the donor or recipient. Other sample sources include, for example, saliva, the fluid in which an organ or tissue for transplant is stored prior to transplant, or small biopsies of the tissue itself. When tissue biopsies are used, they can be homogenized, for example in phosphate buffered saline (PBS) or, alternatively, in a detergent-containing buffer to solubilize the polypeptides to be detected.

Apparatus:

An apparatus for assessing the success of a transplant includes an array platform to hold surface chemistry or a capture agent necessary to bind a plurality of different biomarkers from a sample, a detection mechanism to determine the quantity and/or type of biomarkers bound to the platform, a processor including a comparison mechanism for comparing biomarker detection data from the sample with a reference and a mechanism for determining the condition of the transplant tissue based on the comparison of biomarker detection data from the sample with the reference.

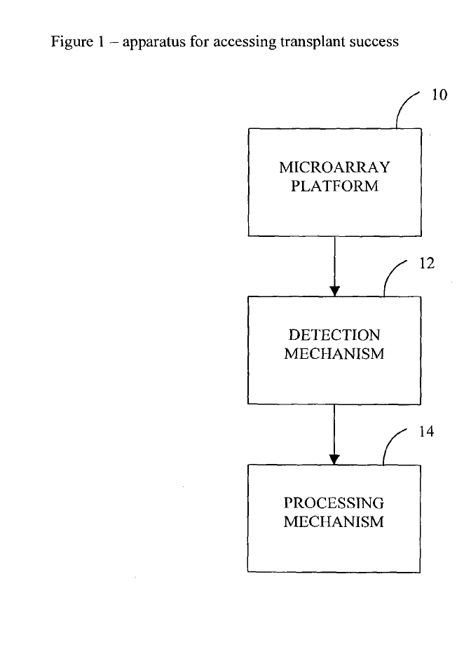

In the embodiment shown in FIG. 1, the biomarkers may be DNA, RNA, or polypeptides. When the biomarkers are polypeptides, protein microarray technology is used to detect proteins in a sample and monitor their expression levels in the sample. A microarray platform 10 uses a capture array of antibodies to detect the target proteins in the sample. When the biomarkers are DNA or RNA, a nucleic acid array is alternatively used.

A detection mechanism 12 is used to determine the quantity and/or type of the target polypeptides in the sample that are bound to the platform. The detection mechanism can be one of a number of options described herein below.

A processing mechanism 14 processes the data gathered by the detection mechanism 12 to assess the success of a transplant of a cell, tissue, or organ. The processing mechanism 14 compares the data from the sample with a reference, and determines the condition of the cell, tissue, or organ to be transplanted based on the comparison of polypeptide detection data from the sample with the reference. Based on the presence, absence or relative amount of biomarker polypeptides, a treatment determination can be made before and after the transplant of the cell, tissue, or organ.

Surface Chemistry:

The role of a given surface chemistry agent or capture agent is to bind one or more biomarkers present in a sample from a transplant donor or recipient or from the cell, tissue or organ itself. Once bound, the biomarkers can be detected to generate a profile or spectrum of the biomarkers present and to facilitate comparison of the profile, which in turn permits assessment of the status of the transplant.

The platform surface can include any of a number of different materials, including, for example, glass, ceramic, silicon wafer, metals, organic polymers, and beads (porous or non-porous) of cross-linked polymers (e.g., dextran, agarose, etc.) or metal. A glass, silicon or metal surface is preferred. A surface can be coated with a material, for example, gold, titanium oxide, silicon oxide, etc. that allows derivatization of the surface.

When the surface is a bead, the bead can be marked with one or more different fluorescent dyes, each dye corresponding to a particular capture agent. A sample is then exposed to a mixture of these coded beads. For polypeptide biomarkers, this permits simultaneous measurement of different proteins in a single sample volume. One detection method that may be used here is flow cytometry. A further alternative is the use of “barcoded” nanoparticles, as described by Walt et al., 2000, Science 287: 451-454; Battersby et al., 2000, J. Am. Chem. Soc. 122: 2138-2139; Bouchez et al., 1998, Science 281: 2013-2016; and Han et al., 2001, Nature Biotechnol. 19: 631-635. These nanoparticles have “stripes” of different metals that vary in number and width, permitting a broad range of different detectable combinations of particles, each derivatized with one or more different capture agents. Detection of proteins bound to nanoparticles can be performed using, for example, mass spectrometry or fluorescence.

Where necessary, the surface for the array can be derivatized with a bifunctional linker that binds a capture agent to the surface. A bifunctional linker generally has a functional group that can covalently bind with a functional group on the surface and a functional group that binds or can be activated to bind a capture agent. Examples of bifunctional linkers include aminoethyl disulfide and aminopropyl triethoxysilane. Alternatively, capture agents can be bound to the surface non-covalently through hydrophobic, van der Waals or ionic interactions.

A number of capture agents that bind proteins are known in the art. These include, for example, antibodies, which can be bound to a surface by any of a number of means that are well known in the art. The term “antibodies” as used herein encompasses any reactive fragment or fragments of antibodies such as Fab molecules, Fab proteins, single chain polypeptides, or the multi-functional antibodies having binding affinity for an antigen. The term includes chimeric antibodies, altered antibodies, univalent antibodies, bi-specific antibodies, monoclonal antibodies, and polyclonal antibodies.

An array can include separate spots of individual antibodies specific for known target proteins. If desired, separate spots can alternatively include more than one antibody, such that a spot can bind two or more known proteins. A variety of different antibodies are commercially available, and those of ordinary skill in the art can raise additional antibodies through standard methods. Spots of antibodies or any other capture agent can be arranged on the surface in a linear array, or, for example, in a grid arrangement that can be accessed by a detection device. Generally, any arrangement of spots that is compatible with a given detection device can be used. Arrays will include at least two spots comprising capture agent(s), and preferably more, e.g., 5, 10, 20, 50, 100, 250, 500 spots or more.

Additional capture agents include, for example, ion exchange and reversed-phase affinity surfaces that interact with moieties on the protein targets. A number of different surface chemistry capture agents are available in an array format on chips from Ciphergen (Fremont, Calif.). For example, carboxylate chemistry provides a negatively charged weak cation exchanger in the CM10 and WCX2 chips, and the SAX2 chip uses quaternary amine functionality for strong anion exchange. Ciphergen also sells chips with immobilized metal affinity capture agent (IMAC3), an agent that mimics reversed-phase chromatography with C16 functionality (H4), and an agent that binds through reversed-phase or hydrophobic interactions (H50), among others. Each of these agents will bind different proteins in a sample with varying degrees of selectivity. In one aspect, a single chip can have a plurality of spots with different capture agents, such that a different subset of proteins in a sample will bind to each different capture agent.

When a protein-containing sample, e.g., urine or serum, is contacted with a surface bearing a capture agent that binds proteins in that sample, proteins bind the capture agent and unbound proteins can be removed by washing. The removal of unbound proteins and other substances reduces the complexity of the sample and the resulting protein profile.

The capture agents or surface chemistry that is used for DNA/RNA microarray are preferably complementary sequences that bind the DNA/RNA. Some of these sequences include, but are not limited to, oligonucleotides, cDNA or PCR fragments of mRNA. Collectively, these are called probes and are also preferably tagged with a reporter, including, but not limited to, an isotope or a fluorophore.

Detection Mechanisms:

In one aspect, the detection mechanism involves Surface Enhanced Laser Desorption/Ionization coupled with Time of Flight mass spectrometry, or SELDI-TOF. SELDI is described in U.S. Pat. Nos. 5,719,060, 6,020,208, 6,027,942 and 6,124,137 which are incorporated herein by reference. The basic principle of SELDI-TOF is that a protein bound to a surface is bombarded with laser energy which induces its desorption from the surface and ionization. The time of flight of the ionized protein to a detector is recorded and converted to protein molecular weight (larger polypeptides generally have longer flight times). The amount and molecular weight of numerous proteins present in a sample can be detected simultaneously to generate a profile or spectrum of the proteins in the sample. With TOF-mass spectrometry, one can obtain information on hundreds or thousands of different proteins or peptides at a single site on an array. The method is capable of detecting nanomole to sub-femtomole quantities of protein on a spot, corresponding to millimolar to picomolar concentrations in a biological sample. Comparison of the profiles from different samples will permit the identification of protein differences between the samples, and the differences permit the assessment of the status of a transplant.

A SELDI-TOF device, the ProteinChip® Reader, is commercially available from Ciphergen (Fremont, Calif.). That device can be used essentially according to the manufacturer's instructions to generate protein profiles for samples from a transplant donor, recipient or tissue. However, exemplary conditions are as follows: The instrument can be operated in the positive ion mode with a source and detector voltage of 20 and 1.8 kV, respectively. Time-lag focusing can be used, e.g., with a pulse voltage and lag time of 3000 V and 673 ns, respectively. Laser intensity is set at 150 (approximately 100 μJ) using a nitrogen laser emitting at 337 nm. The digitizer operates at 250 mHz. The laser traverses 66% of the target area in a linear sweep to generate each spectrum (von Eggeling et al., 2000, BioTechniques 29: 1066-1070).

The apparatus disclosed herein also includes a processor including a comparison mechanism for comparing polypeptide detection data from a sample with a reference. Software for comparison of spectra are available in the art. For example, Ciphergen (Fremont, Calif.) sells a software package, ProteinChip® Software 3.0, designed for use with its ProteinChip® Reader that performs comparisons of the mass spectra and will identify peaks that differ between samples. Analysis software and protein array chips are also available from LumiCyte (Fremont, Calif.). Software designed for interpretation and comparison of mass spectrometry data is also available from, for example, ChemSW, Inc. (N. Fairfield, Calif.), Scientific Instrument Services (Ringoes, N.J.), Agilent Technologies (Palo Alto, Calif.), BioBridge Computing (Malmo, Sweden), and Bioinformatics Solutions (Waterloo, Ontario).

Alternatives to mass spectrometric detection include fluorescent detection. WO 0004382, incorporated herein by reference, describes an ELISA-based strategy in which antibodies are arrayed on a chip and binding of protein antigen is detected by fluorescence, phosphorescence or luminescence. Labeled secondary antibodies can be employed in this or other aspects of the detection method.

Another alternative for the detection of bound proteins is surface plasmon resonance, which detects binding events by using changes in the refractive index of a surface caused by increases in mass. This approach is particularly appropriate when specific capture agents, e.g., antibodies, are used.

Additional detection alternatives include resonance light scattering (equipment and methods provided by Genicon Sciences, Carlsbad, Calif.) and atomic force microscopy (BioForce Laboratories, Ames, Iowa).

The most common probes for DNA/RNA are fluorescent dyes. The detection mechanism when using fluorescent dyes is preferably a laser that stimulates the excitation of the fluorophor at a specific wavelength. A detector with a filter for a specific emission wavelength then gauges the intensity of the light emission. For isotope labeled probes, a Beta counter is used, or the array can be exposed to film and the intensity of the developed “spots” can be calculated using image analysis software and transillumination.

Profiles/Biomarker Difference Maps

The pattern of the presence and/or amount of a plurality of biomarkers in a given sample forms a biomarker profile for that sample. A comparison of the profiles from samples taken at various times before and after transplantation and in successful and ultimately unsuccessful transplants permits the creation of a biomarker difference map for a given cell, tissue or organ.

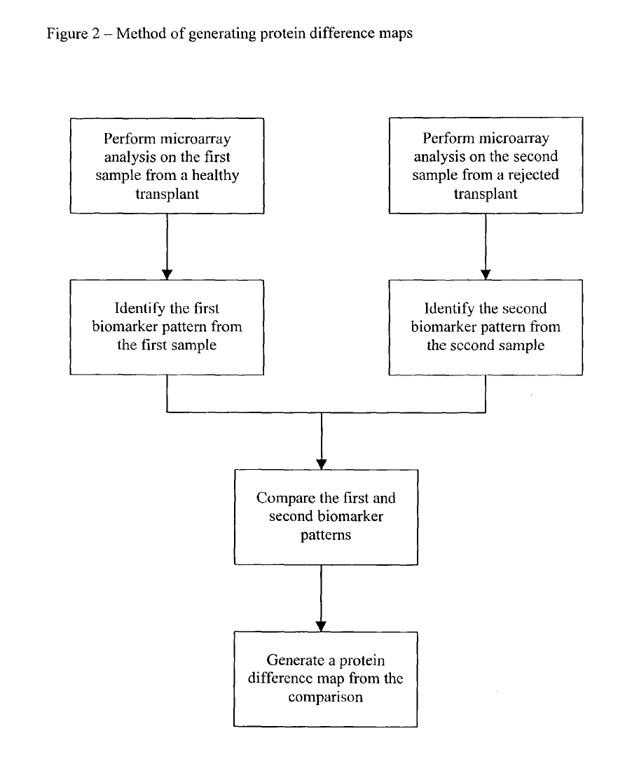

As an example, FIG. 2 shows a protein difference map generated by identifying a biomarker pattern for a cell, tissue or organ, and comparing it to the biomarker pattern for a cell, tissue or organ at a different stage of transplantation (e.g., differing times pre-transplant, differing times post-transplant, or from an individual undergoing different degrees or stages of transplant failure or rejection). The present invention may be used to create a database of biomarkers correlated with an array of clinical outcomes. The database relates biomarker expression across varied stages of transplantation to known clinical outcomes for each donor/recipient. These data collectively generate the reference pattern for any future transplant at any stage. The more data points in the database, and the more correlations between biomarker expression and outcome, the stronger the statistical correlation. One could obtain data at successive timepoints for successful transplants, and data at the same, successive timepoints for unsuccessful transplants. The protein difference map takes note of those proteins that appear or disappear or that increase or decrease in abundance in healthy versus ultimately unhealthy transplants. The protein difference map can also take note of trends in the amount of individual biomarkers, rather than absolute amounts of the biomarkers, that correlate with the outcome of the transplant.

Data Analysis and Decision Making Based on Profiles:

Data obtained from a biomarker array can be analyzed manually if needed, but are preferably analyzed by computer. Generally, any detection method for a biomarker array as described herein will generate a readout that can be stored and analyzed in digital form. For example, computer data acquisition from fluorescence detectors and from mass spectrometry devices is well known in the art.

As noted above, software for comparison and analysis of protein detection data are available in the art. For example, Ciphergen (Fremont, Calif.) sells a software package, ProteinChip® Software 3.0, designed for use with its ProteinChip® Reader that performs comparisons of the mass spectra and will identify peaks that differ between samples. Software designed for interpretation and comparison of mass spectrometry data is also available from, for example, ChemSW, Inc. (N. Fairfield, Calif.), Scientific Instrument Services (Ringoes, N.J.), Agilent Technologies (Palo Alto, Calif.), BioBridge Computing (Malmo, Sweden), and Bioinformatics Solutions (Waterloo, Ontario). Similar software products are also available for the analysis of readouts from fluorescence detectors or other detection devices.

Data obtained from a DNA/RNA array can also be analyzed manually if needed, but with thousands of gene sequences per chip, this can be very time consuming. The data generated can be stored in what are collectively called microarray databases, which are repositories that store the measurement data, manage a searchable index, and make the data available to other applications for analysis and interpretation either directly, or via user downloads. Software is also available for the analysis of DNA/RNA microarray database information. For example, ArrayTrack (NCTR/FDA) is a free bioinformatics resource that allows for the management, analysis, and interperetation of “omics” data. Additionally, there are almost 50 microarray analysis software applications available on the world-wide web at The Gene Ontology (http://www.geneontology.org/GO.tools.microarray.shtml, herein incorporated by reference).

As noted above, software for comparison and analysis of protein detection data are available in the art. For example, Ciphergen (Fremont, Calif.) sells a software package, ProteinChip® Software 3.0, designed for use with its ProteinChip® Reader that performs comparisons of the mass spectra and will identify peaks that differ between samples. Software designed for interpretation and comparison of mass spectrometry data is also available from, for example, ChemSW, Inc. (N. Fairfield, Calif.), Scientific Instrument Services (Ringoes, N.J.), Agilent Technologies (Palo Alto, Calif.), BioBridge Computing (Malmo, Sweden), and Bioinformatics Solutions (Waterloo, Ontario). Similar software products are also available for the analysis of readouts from fluorescence detectors or other detection devices.

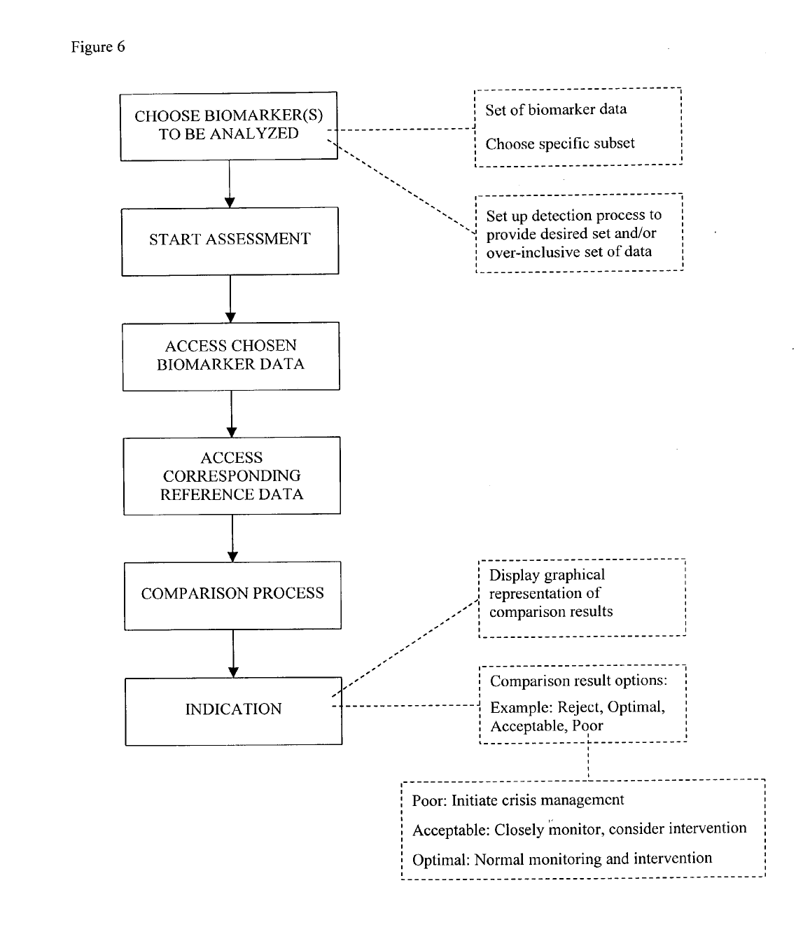

FIG. 6 shows a schematic diagram of a process performed by a computerized system for identifying the condition of a cell, tissue or organ that is being considered for transplant. A set of stored biomarker data for the cell, tissue or organ to be assessed, or a specific subset of stored biomarker data is chosen. This can include the set-up of a detection process to provide the desired set of data and/or an overinclusive set of data. Once assessment is started, the chosen biomarker data to be assessed are accessed, corresponding reference data are accessed, the biomarker data to be assessed is compared to the reference data, and an indication of the condition of the cell, tissue or organ is graphically displayed, based on the comparison. The system can also make a suggestion regarding transplant or post-transplant treatment approach, including a suggestion to proceed or not proceed with the transplant, a suggestion to proceed with the transplant with heightened monitoring for one or more indicators of potential problems, a suggestion to consider drug intervention for the transplanted material, or a suggestion to initiate aggressive drug intervention for the transplanted material. The suggestions are based on the comparison of biomarker data to be assessed and reference biomarker data in light of the known outcome of treatment for the reference biomarkers.

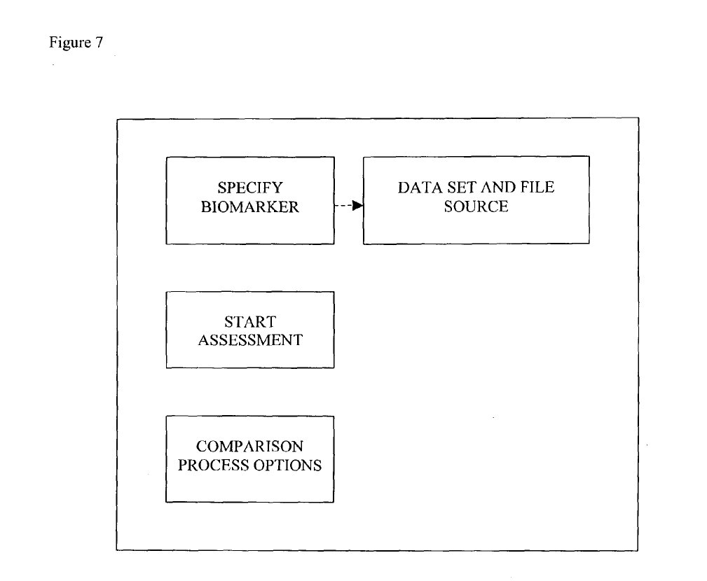

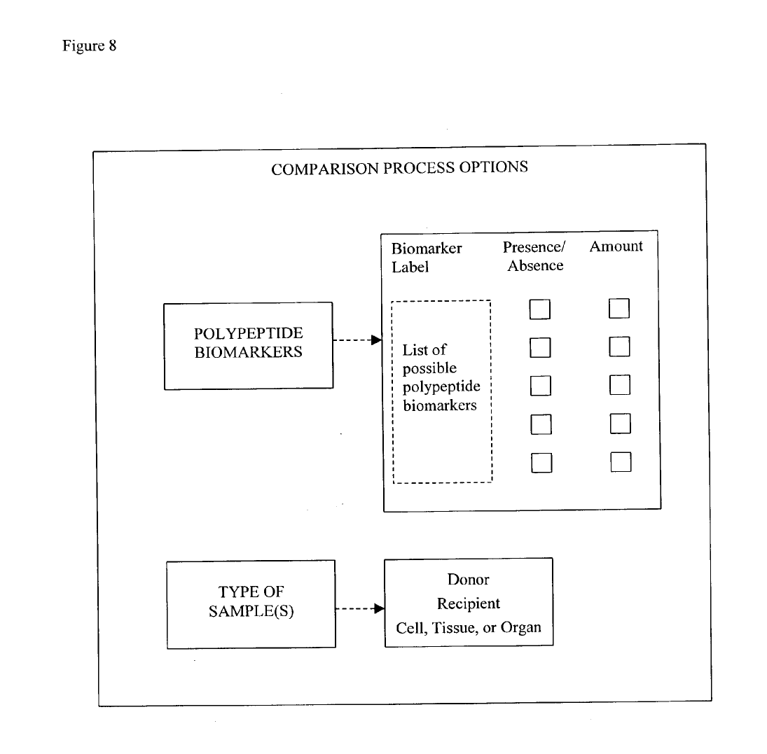

FIG. 7 shows a schematic of a computer display screen shot including a graphic representation of buttons to specify biomarker(s) to be assessed, start assessment and set comparison process options. Clicking on the “specify biomarkers” button brings up a menu permitting selection of data set and file source for the selected biomarker(s). Clicking on the “start assessment” button begins process shown in FIG. 6, which includes the comparison of the biomarker data to be assessed and reference biomarker data. Clicking on the “comparison process options” button brings up a menu for selection of options (see FIG. 8).

FIG. 8 shows a schematic of a computer display screen shot displaying comparison process options. Clicking on the “polypeptide biomarkers” button brings up a menu permitting a choice of biomarkers, with a further choice (check boxes) for each as to whether one wants to compare “Presence/Absence” or “Amount” of the biomarker, or both. Clicking on the “Type of Sample” button brings up a menu permitting a choice of biomarker data from transplant donors, transplant recipients, or transplant cells, tissues or organs themselves.

As discussed above, “a difference between the pattern observed for a transplant and a reference pattern” encompasses both similarities and differences between biomarker patterns. Thus, when there is no difference or very little difference between a reference pattern and a test sample pattern, the “difference” is indicative that the transplant outcome for the test sample will be similar to the outcome for the reference sample(s). Alternatively, where there is a wide “difference” (e.g., 50% or more higher or lower than the reference), the outcome of the test sample transplant will likely differ from the outcome of the reference pattern sample(s).

When a transplant donor or recipient sample shows a level or trend of one or more biomarkers that correlates with a level on a difference map that in turn correlates with a present or potential future problem with the transplant, treatment decisions can be guided by that information. Thus, a mechanism that determines the condition of a cell, tissue or organ before or after transplant involves a comparison of the biomarker profile from that cell, tissue or organ with a reference profile or database of profiles. Thus, a level of one or more biomarkers for a pre-transplant tissue or organ that correlates with a poor post-transplant prognosis could guide a decision not to transplant that organ.

Alternatively, a level or pattern of one or more biomarkers for a post-transplant tissue or organ that correlates with a poor post-transplant prognosis can guide a decision to aggressively treat with drugs that would otherwise not be preferred. Post-transplant monitoring of biomarkers as described herein will also permit the detection of changes in biomarkers within the recipient that herald future problems with the transplant. Because the procedure is relatively non-invasive (preferably using urine or blood testing) and because the detection is rapid (particularly when SELDI-TOF is used), the methods described herein are well suited to ongoing post-operative monitoring of transplanted tissue. As noted, software for comparison of biomarker profiles obtained by SELDI-TOF is available from Ciphergen. Software packages suitable for the analysis of profile data obtained in other ways is known to those skilled in the art and will frequently be included with a detection device.

### EXAMPLES

### Analysis of Biomarkers in Renal Transplant

Renal Preservation Solutions Collection

Following standard porcine nephrectomy, kidneys were gently flushed through the renal artery with HypoThermosol®-FRS (HTS-FRS or HTS) hypothermic storage solution (BioLife Solutions, Inc., Bothell, Wash.) at 4° C. Following flushing, kidneys were perfused with and submerged in HTS-FRS and statically stored at 4° C. for 6 days, which is well beyond the current acceptable preservation interval of 2-3 days. During preservation, kidneys were flushed with fresh HTS-FRS every 24 hours and the effluent solution was collected during the flush procedure and stored at −80° C. for analysis.

Urinary Analysis from Transplant Recipients

Urine samples were collected from human donor and recipient patients following renal transplant at 24, 48, and 72 hours post transplant following standard biologic fluid collection NYPIRB protocol. Following collection, cells secreted into the urine were collected by centrifugation and frozen at −80° C. Upon thawing, cells were lysed in RIPA buffer (20 mM Tris (pH 8.0), 137 mM NaCl, 10% glycerol, 1% Nonidet P-40, 0.1% SDS, 0.5% deoxycholate, 2 mM EDTA) supplemented with protease inhibitors (5 mM benzamidine, 1 mM PMSF, 20 uM Pepstatin A, 7.5 mM EDTA). Cell lysate was centrifuged at 14,000 rpm for 10 minutes at 4° C., and the supernatant (cytosolic protein) was separated and stored at −20° C.

SELDI-TOF Protein Analysis

Protein Standards

Insulin and Glucagon standards were obtained from Santa Cruz Biotechnology (Santa Cruz, Calif.). Indicated amounts of protein standard were analyzed using an NP1 chip array following standard manufacturer instructions.

Sample Protein Analysis

Preservation solution analysis was performed on the HTS collected during cold storage of porcine kidney utilizing a Ciphergen Weak Cationic Exchange chip array (WCX2). The WCX2 chip bioprocessor technique was utilized to enhance protein capture from a diluted sample. Ten microliters per HTS sample was used on each chip array spot. Analysis of urine samples from transplant patients was performed on cellular protein extracts (1 μg/spot) using Ciphergen Normal Phase chip arrays (NP1). Preparation and analysis of the chips was performed following the manufacturer's standard protocol. Briefly, samples were applied to their respective chip surface spots and allowed to bind. Subsequent to the binding interval, excess unbound protein was washed off the chip with binding buffer and allowed to air dry. Following drying, Energy Absorbing Molecule (EAM) was added to each sample spot and allowed to dry again. Protein samples were then analyzed using the Ciphergen ProteinChip Reader in which sample proteins were desorbed by laser activation and time-of-flight (TOF) was recorded and converted into protein molecular weight. Protein spectra are resultant from 10-20 ProteinChip® scans from each sample spot.

Data Analysis

Protein profiles from samples obtained from the SELDI-TOF ProteinChip® system were individually analyzed for peak identification and intensity using the Ciphergen Peaks software (version 2.0). Intensity data from corresponding individual peaks from multiple samples were combined to determine average peak intensity (+/−SEM). Data on protein profiles from preservation flush solutions was collected from samples obtained from three separately preserved porcine kidneys from three separate individual animals. Urine sample were provided gratis by Columbia University and the data reported represents average protein profiles and intensities (+/−SEM) from three individuals. Analysis of statistical significance was performed using single-factor ANOVA and P-values are reported in the text.

Results

Characterization of SELDI ProteinChip® samples

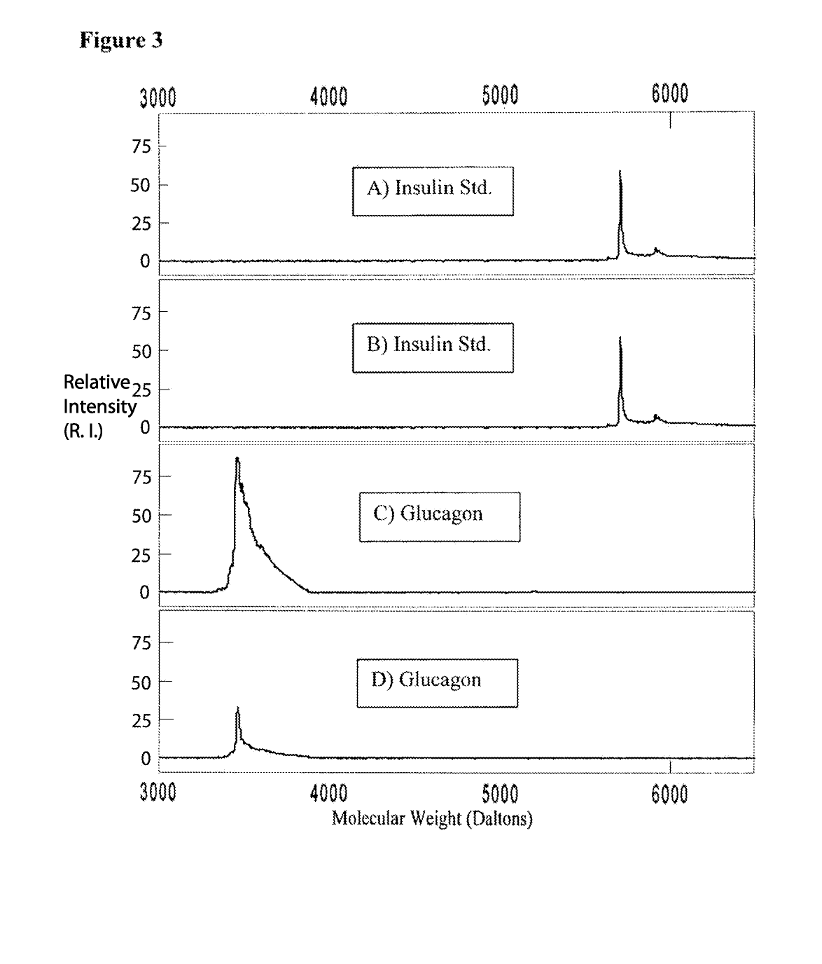

SELDI ProteinChip® calibration and standardization was performed using purified protein standards. Purified Insulin and Glucagon samples were analyzed with the system to determine their molecular masses and compared with their reported predicted molecular masses (FIG. 3). Analysis of the Insulin standard consistently yielded a distinctive peak at 5752 Da (spectra from two samples shown in FIG. 3, Spectra A and B), which closely resembled the reported molecular mass (5807 Da). Similar analysis was performed using a Glucagon standard to assess calibration at multiple molecular masses and yielded a molecular mass of 3460 Da (spectra from two samples shown in FIG. 3, Spectra C and D), which again resembled that of the predicted mass (3482 Da). In addition to molecular mass determination, insulin standard analysis on duplicate chip spots revealed reproducible spectra (P<0.005) (Spectra A and B). Variation of glucagon standard concentration revealed both spectra reproducibility and sensitivity (Spectra C and D). These data revealed that the established protocol enabled reproducible molecular mass determination within 0.7% of predictive values as well as sensitivity for protein concentration comparison between samples.

Analysis of Preservation Medium

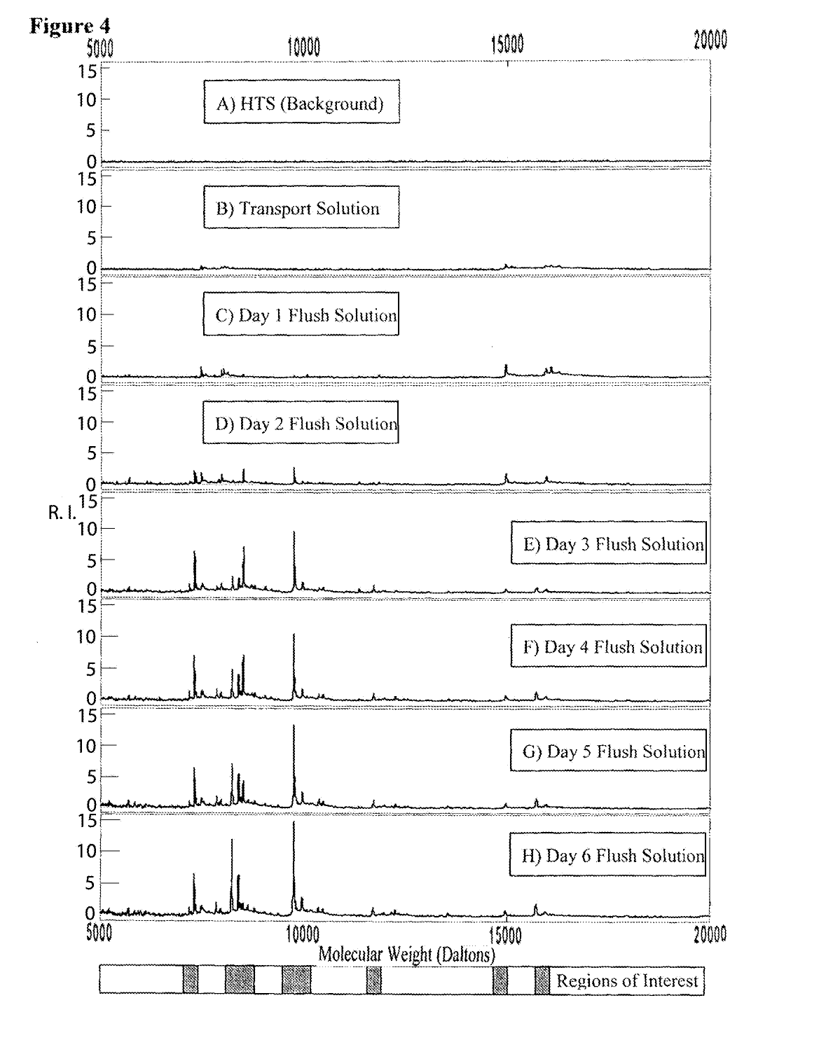

Porcine kidneys were perfused with HypoThermosol®-FRS and statically stored at 4° C. for a period of 6 days. Kidneys were gently flushed daily with fresh HTS and the flush solution was collected for ProteinChip™ analysis (FIG. 4). Analysis of the flush solutions revealed distinct phenomic fingerprints (protein profiles) in the samples characterized by the appearance of an increasing number of unique peaks as well as an increasing intensity of existing peaks. Evaluation of the background level of HTS yielded no discernable peaks (Spectra A). Transport solution analysis [HTS surrounding the kidneys during transport (Day 1)] revealed few minor protein peaks not statistically above background (Spectra B). In comparison, analysis of the HTS within the kidneys during transport on day 1 flush solution resulted in the appearance of several protein peaks ranging in molecular mass from 7350 Da to 15950 daltons (D), with distinct peaks appearing around 7405, 7861, 14952, 15950 Da (Spectra C). Day 2 flush solutions revealed the appearance of 3 new protein peaks at 7317, 8525, and 9758 Da yielding 7 distinct peaks total (Spectra D). At 3 days of storage, the appearance of additional peaks in the flush solution continued, most notably at 8254, 9966, and 11706 Da (Spectra E). Following 4-6 days of storage, no new discernable peaks were noted from those at three days, but there was a significant intensification of the existing peaks each subsequent day of analysis (Spectra F-H). In particular, the intensity of the peak at 9966 Da increased from 10 (Day 3) to 13 (Day 5) to 15 (Day 6) (P<0.01) and the peak at 8254 Da increased from 2 to 7 to 13 over the same interval (P<0.005) on average. Despite the overall trend toward peak intensification, it was observed that the peak at 8525 Da increased from 3 to 7 between day 2 and 3 (P=0.0053) and subsequently decreased to around 5 (P=0.009) at day 5 and was at background levels by day 6 (P=0.12 from background).

Urine Protein Analysis from Transplant Patients

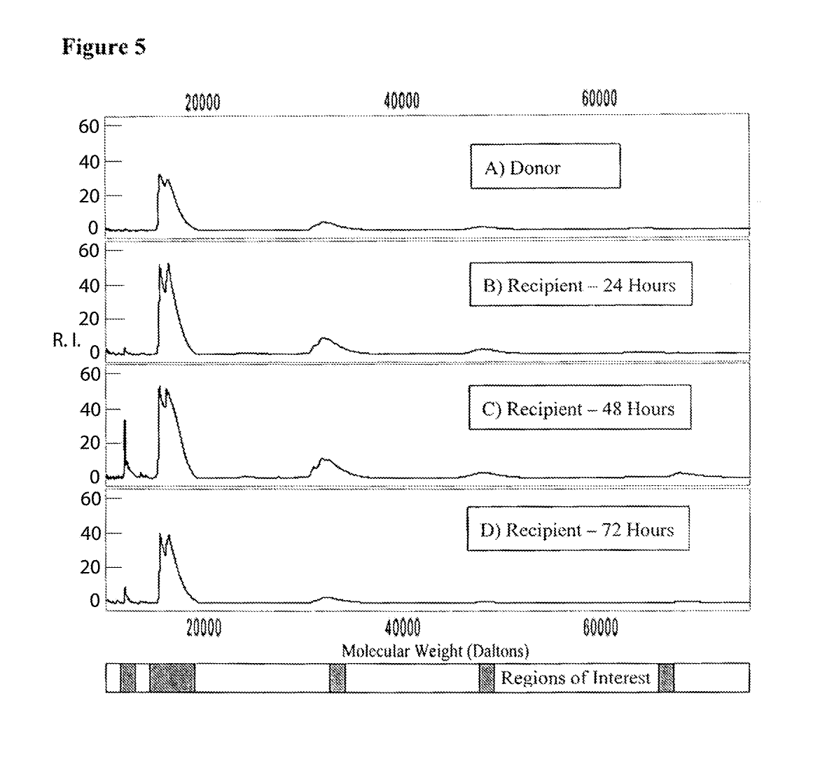

Urine from patients following kidney transplantation was collected daily over a postoperative period of 3 days and analyzed for the presence, concentration, and profile of proteins, and compared to urine protein profiles from the donors (FIG. 5). Profiling of donor urine showed the presence or several proteins, which was represented by the appearance of 4 peaks during SELDI-analysis with molecular masses of 15620, 16394, 47955, and 64005 Da with intensities of 31, 28, 2 and 5, respectively (Spectra A). The 64005 Da protein was present in both a 1H⁺ and 2H⁺ form resulting in an additional peak at an apparent molecular mass of 32560 Da. Analysis of recipient urine 24 hours following transplantation revealed intensification in proteins concentration above that observed in the donor urine (Spectra B). Twenty-four hour sample analyses revealed peak intensities of 50, 54, 5, and 10 for the peaks with molecular masses of 15620, 16394, 47955, and 64005 Da, respectively. The observed changes represent significant increases in protein concentration when compared to their respective peaks from the donor sample (P<0.0064). In addition to the increase, there was also the appearance of an additional peak at 11997 Da with an intensity of approximately 2. Continued analysis of recipient urine at 48 hours post-transplant revealed a continued trend of increasing intensity in the 11997 Da and 64005 Da proteins form the 24 hour sample from 2 to 32 (P<0.001) and 10 to 12 (P=0.008), respectively (Spectra C). Peaks at 15620 Da and 16394 Da appeared to maintain a relatively consistent intensity over the 24 to 48 hour interval with average intensities ranging between 50-54 (P>0.27). As with the 24-hour sample, there was the appearance of a unique peak at 67919 Da with an average intensity of 3 in the 48-hour post-transplant samples. Urine samples collected 72 hours post-op from recipients showed a decrease in peak intensity for all identified proteins (Spectra D). On average, all protein peaks returned to that of donor levels by 72 hours post-op (P>0.039) with the exception of the peak at 11997 Da which decreased significantly from 48 hour samples from 32 to 8 (P<0.001), while remaining above that of donor levels (P=0.004).

The present invention permits the analysis of phenomic fingerprints present in preservation solutions prior to transplantation, and in patient urine samples following transplantation, were performed. Changes to a cell, tissue or organ during hypothermic storage can be assessed and monitored through analysis of proteins released from the tissue during the preservation interval. Specifically, during storage, cellular degradation results in the release of proteins into the preservation medium, and the level and profile of these proteins can serve as an indicator for organ quality. These data also demonstrate protein profiling of urine samples from transplant recipients as a means for implant and patient monitoring.

Through the utilization of SELDI-ProteinChip® microarray technology, high-throughput protein analysis allows for the identification of unique expression profiles from individual preservation solution samples. Analysis of flush solutions from kidneys stored at 4° C. for 6 days and collected at 24 hour intervals revealed an increase in the amount and diversity of proteins released during preservation. The appearance and increase in the concentration of certain proteins in the preservation solution is believed to be a result of tissue degradation, and contains biomarkers, which serve as indicators of organ status. In particular, the significant increase in protein concentration (peak intensity) and appearance of a number of additional proteins, as discovered in the 3 day preservation solution samples in this study, represent a significant diagnostic indicator of organ transplant quality. When one considers the present generally accepted 24 to 48 hour preservation interval for kidneys, this alteration in the phenomic fingerprint may represent a significant early indicator. The analysis of preservation solution phenomic fingerprints, when correlated with transplant procedural and post-operative data, can serve as pre-operative tissue diagnostic and procedural success predictive indicator.

The need for the development of rapid, high-throughput, real-time analytical tools and procedures will prove critical to the continued evolution of the surgical field of transplantation. In one embodiment, the present invention uses SELDI-TOF microarray technology for the analysis of pre-implantation organ quality. The application of SELDI-TOF microarray technology allows for 1) the rapid and accurate determination of phenomic fingerprints from complex biological samples, 2) phenomic fingerprints can serve as quantitative diagnostic indicators of organ quality, 3) analysis of urine for protein profiles represents a significant source of information regarding patient post-operative status, and 4) utilization of phenomic profiling and microarrays may facilitate the identification of specific biomarkers to serve as real-time predictive indicators for transplantation efficacy.

Accordingly, it is to be understood that the embodiments of the invention herein described are merely illustrative of the application of the principles of the invention. The various features of the embodiments of the present invention can be combined within the spirit of the present invention. Reference herein to details of the illustrated embodiments is not intended to limit the scope of the claims, which themselves recite those features regarded as essential to the invention.

1-31. (canceled)

32. A method of identifying a biomarker that will aid in predicting an outcome of a potential transplant of a cell, tissue or organ into a first transplant recipient, comprising the steps of:
a) evaluating at least one sample associated with an actual transplant at a plurality of timepoints after transplantation;
b) identifying at least one biomarker in the sample;
c) identifying at least one biomarker in at least one pre-transplant sample of a preservation solution in which the cell, tissue, or organ is stored; and
d) correlating the biomarker with at least one clinical outcome for the transplantation.

33. (canceled)

34. The method of claim 32, wherein the sample associated with an actual transplant is selected from the group consisting of at least one post-transplant patient sample; body fluids; biopsies and any combination of post-transplant patient samples, body fluids, and biopsies.

35. The method of claim 32, wherein the timepoints in step a) are chosen in a range of 1 minute to a month from a date of the transplant.

36. The method of claim 32, wherein the timepoints in step a) comprise taking a sample at least once each day for a time period until a second transplant recipient of the actual transplant is discharged from a medical facility.

37. The method of claim 32, wherein step b) comprises the substep of using a SELDI TOF array to identify the biomarker in at least one pre-transplant sample of preservation solution.

38. The method of claim 32, wherein the biomarkers comprise at least one DNA, RNA, or peptide biomarker.

39. The method of claim 38, wherein the biomarker is a biomarker associated with apoptosis or necrosis.

40. A method of identifying a biomarker that will aid in predicting an outcome of a transplant of a cell, tissue, or organ into a transplant recipient, comprising the steps of:
a) evaluating a plurality of samples from a plurality of transplants;
b) identifying at least one biomarker in the samples; and
c) correlating the biomarker with at least one clinical outcome of the transplant;
wherein the samples are samples taken from a preservation solution in which the cell, tissue or organ was stored prior to the transplant.

41. (canceled)

42. The method of claim 40, wherein the biomarker comprises at least one DNA, RNA, or peptide biomarker.

43. The method of claim 42, wherein the biomarker is a biomarker associated with apoptosis or necrosis.

44. The method of claim 32, further comprising the steps of repeating steps a), b), and d) and creating a reference pattern of a plurality of biomarkers for the cell, tissue or organ using data obtained in step d).

45. The method of claim 44, further comprising the step of comparing the biomarker identified in the sample in step c) with the reference pattern.

46. The method of claim 45, further comprising the step of determining the medical condition of the potential transplant from a difference between a pattern observed for said transplant and the reference pattern.

47. The method of claim 44 wherein the reference pattern of a plurality of biomarkers comprises a biomarker difference map generated by a method comprising the steps of:
i) performing analysis of a first sample from a cell, tissue, or organ from a first actual transplant recipient having a positive clinical outcome;
ii) identifying a first biomarker pattern from the first sample;
iii) performing analysis of a second sample from a cell, tissue, or organ from a second actual transplant recipient having a negative clinical outcome;
iv) identifying a second biomarker pattern from the second sample;
v) comparing the first and second biomarker patterns;
vi) optionally repeating one or more of steps (i) through (v) with additional samples; and
vii) generating a biomarker difference map from the comparison.

48. The method of claim 47, wherein the biomarker difference map comprises a protein difference map.

49. The method of claim 47 wherein the positive clinical outcome comprises the first actual transplant recipient having a healthy transplant and the negative clinical outcome comprises the second actual transplant recipient having a rejected transplant.

50. The method of claim 32, wherein a source of the pre-transplant sample in step c) comprises preservation solution flushed from the cell, tissue, or organ.

51. The method of claim 40, further comprising the step of creating a reference pattern from the data obtained in steps a) through c) for a plurality of biomarkers for the cell, tissue or organ from transplant recipients for which a clinical outcome is known.

52. The method of claim 51, further comprising the step of comparing a pattern of the plurality of biomarkers from a tissue matched cell, tissue or organ to be transplanted to the reference pattern.

53. The method of claim 52, further comprising the step of determining the medical condition of the transplant from a difference between the pattern observed for said transplant and the reference pattern.

54. The method of claim 51 wherein the reference pattern of a plurality of biomarkers comprises a biomarker difference map generated by a method comprising the steps of:
i) performing analysis of a first sample from a cell, tissue, or organ from a first transplant recipient having a positive clinical outcome;
ii) identifying a first biomarker pattern from the first sample;
iii) performing analysis of a second sample from a cell, tissue, or organ from a second transplant recipient having a negative clinical outcome;
iv) identifying a second biomarker pattern from the second sample;
v) comparing the first and second biomarker patterns;
vi) optionally repeating one or more of steps (i) through (v) with additional samples; and
vii) generating a biomarker difference map from the comparison.

55. The method of claim 54, wherein the biomarker difference map comprises a protein difference map.

56. The method of claim 54 wherein the positive clinical outcome comprises the first transplant recipient having a healthy transplant and the negative clinical outcome comprises the second transplant recipient having a rejected transplant.

57. The method of claim 54 wherein the reference pattern comprises at least one of:
a) biomarkers present in the preservation solution that correlate with post-transplant difficulties; and
b) biomarkers present in the preservation solution that correlate with post-transplant success.

58. The method of claim 40, wherein the samples comprise preservation solution flushed from the cell, tissue or organ.

59. A method of identifying a biomarker that will aid in predicting an outcome of a potential transplant of a cell, tissue or organ into a first transplant recipient, comprising the steps of:
a) evaluating at least one sample associated with an actual transplant at a plurality of timepoints after transplantation;
b) identifying at least one biomarker in the sample;
c) identifying at least one biomarker in at least one pre-transplant sample of a flush solution used to flush the cell, tissue or organ; and
d) correlating the biomarker with at least one clinical outcome for the transplantation.

60. The method of claim 59, further comprising the steps of flushing the cell, tissue, or organ to be transplanted before preserving the cell, tissue, or organ in a preservation solution and collecting an effluent solution of the flush solution to use as the pre-transplant sample.

61. The method of claim 59, further comprising the steps of flushing the cell, tissue, or organ to be transplanted during preservation and collecting an effluent solution of the flush solution to use as the pre-transplant sample.

62. The method of claim 59, wherein the pre-transplant sample comprises preservation solution flushed from the cell, tissue, or organ.

63. A method of identifying a biomarker that will aid in predicting an outcome of a transplant of a cell, tissue, or organ into a transplant recipient, comprising the steps of:
a) evaluating a plurality of samples from a plurality of transplants;
b) identifying at least one biomarker in the samples; and
c) correlating the biomarker with at least one clinical outcome of the transplant;
wherein the samples are samples taken from a flush solution used to flush the cell, tissue or organ.

64. The method of claim 63, further comprising the steps of flushing the cell, tissue, or organ to be transplanted before preserving the cell, tissue, or organ in a preservation solution and collecting an effluent solution of the flush solution to use as the samples.

65. The method of claim 63, further comprising the steps of flushing the cell, tissue, or organ to be transplanted during preservation and collecting an effluent solution of the flush solution to use as the samples.

66. The method of claim 63, wherein the source of the samples comprises preservation solution flushed from the cell, tissue, or organ.
